(19) United States
(12) Patent Application Publication
(10) Pub. No.: US 2025/0000461 A1
(43) Pub. Date: Jan. 2, 2025

(54) SYSTEM AND METHOD FOR DETECTING HEALTH EVENTS

(71) Applicant: Apple Inc., Cupertino, CA (US)

(72) Inventors: Burak Arda OZILGEN, Sunnyvale, CA (US); Gopal VALSAN, Gilroy, CA (US); Chin S. HAN, San Jose, CA (US); Thilaka S. SUMANAWEERA, San Jose, CA (US)

(21) Appl. No.: 18/434,681

(22) Filed: Feb. 6, 2024

Related U.S. Application Data
(60) Provisional application No. 63/511,579, filed on Jun. 30, 2023.

(51) Int. Cl. A61B 5/00; G16H 40/67

(52) U.S. Cl. CPC A61B 5/7282; G16H 40/67; A61B 2560/0247; A61B 2560/0431

(57) ABSTRACT

A system including one or more electronic devices calculates the likelihood of the user of the system experiencing a health event, such as a seizure. In some examples, the system collects data including environmental data and/or physiological data and uses the data to calculate the likelihood of the user of the system experiencing the health event. In some examples, in response to calculating that the likelihood of the user experiencing the health event exceeds a predetermined threshold, the system generates an alert.

### CROSS-REFERENCE TO RELATED APPLICATIONS

This application claims the benefit of U.S. Provisional Application No. 63/511,579, filed Jun. 30, 2023, the entire disclosure of which is incorporated herein by reference in its entirety for all purposes.

### FIELD

This disclosure relates to detecting a likelihood that a user of a system is experiencing or is about to experience a health event.

### BACKGROUND

Some medical conditions can be aggravated by environmental factors, leading to a health event, such as a seizure. Health events can be characterized by a variety of symptoms.

### SUMMARY

This disclosure relates to detecting a likelihood that a user of a system is experiencing or is about to experience a health event. For example, the system includes one or more electronic devices including and/or in communication with one or more sensors. In some examples, the system uses the sensors to collect data including physiological data of the user of the system and/or environmental data about the physical environment of the system. In some examples, the system uses the data to calculate a likelihood that the user is experiencing a health event or is about to experience a health event. For example, the system calculates the likelihood of the user experiencing a seizure. In some examples, in response to calculating that the likelihood exceeds a predefined threshold, the system generates an alert. For example, the alert includes causing the system to output a visual, audio, and/or tactile indication to the user and/or alerting a system used by another user, such as an emergency contact of the user, emergency services, and/or good Samaritans nearby.

### BRIEF DESCRIPTION OF THE DRAWINGS

FIGS. 1A-1D illustrate example electronic devices that can be configured to sense environmental and/or physiological data relevant to prediction of a health event according to some examples of the disclosure.

FIG. 2 illustrates an example system according to some examples of the disclosure.

FIG. 3 illustrates an example method of calculating the likelihood that a user is experiencing or is about to experience a health event according to some examples of the disclosure.

FIG. 4 illustrates an example method of updating an algorithm used to calculate the likelihood that a user of a system is experiencing a health event or is about to experience a health event according to some examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

This disclosure relates to detecting a likelihood that a user of a system is experiencing or is about to experience a health event. For example, the system includes one or more electronic devices including and/or in communication with one or more sensors. In some examples, the system uses the sensors to collect data including physiological data of the user of the system and/or environmental data about the physical environment of the system. In some examples, the system uses the data to calculate a likelihood that the user is experiencing a health event or is about to experience a health event. For example, the system calculates the likelihood of the user experiencing a seizure. In some examples, in response to calculating that the likelihood exceeds a predefined threshold, the system generates an alert. For example, the alert includes causing the system to output a visual, audio, and/or tactile indication to the user and/or alerting a system used by another user, such as an emergency contact of the user, emergency services, and/or good Samaritans nearby.

Some medical conditions can be aggravated by environmental factors, leading to a health event, such as a seizure (e.g., in response to exposure photosensitive stimulus). Some medical conditions, optionally including seizures, can be detected based on deviations in physiological data of a person from baseline physiological data from that person. In some examples, systems including one or more electronic devices can sense data indicative of the health event, including physiological and/or environmental data, and calculate a likelihood of the user of the system experiencing the health event at the time of calculation and/or in the future. For example, the system can detect that the current environment of the system and, therefore, the user, may trigger the user to experience the health event based on sensed data. As another example, the system can detect physiological data of the user that indicates the user is experiencing or is about to experience the health event.

In some examples, the system can output an indication in response to calculating the likelihood of the user experiencing the health event is above a threshold likelihood. In some examples, outputting the indication includes presenting an audio, tactile, and/or visual indication to the user. Such indications can help the user prepare for the health event, leave the environment to attempt to avoid the health event, and/or contact an emergency contact, emergency services, and/or nearby good Samaritans for assistance. In some examples, additionally or alternatively, outputting the indication includes increasing the types of data sensed and/or increasing the frequency of sensing data.

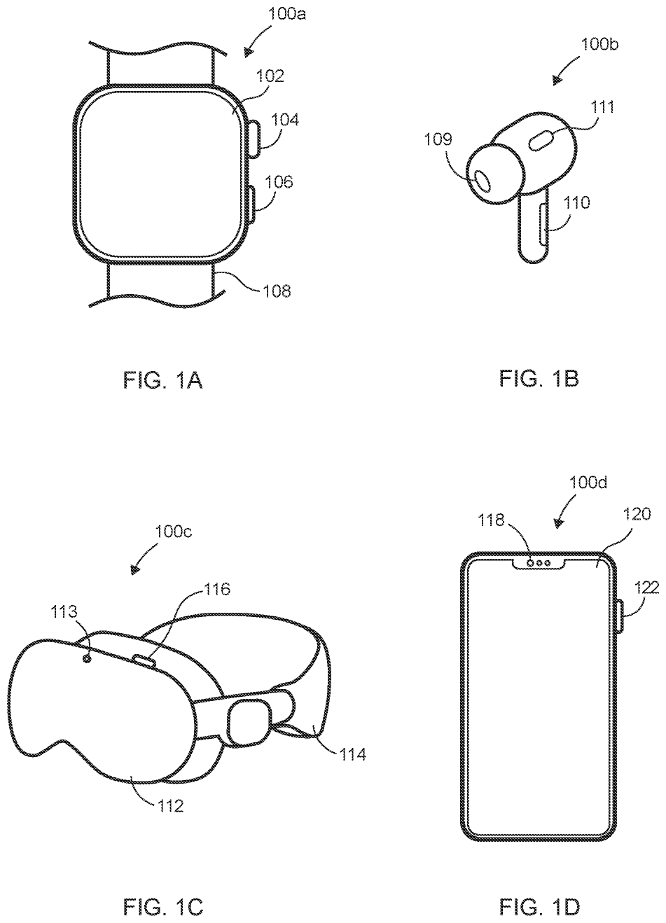

FIGS. 1A-1D illustrate example electronic devices 100a, 100b, 100c, and/or 100d that can be configured to environmental and/or physiological data relevant to prediction of a health event according to some examples of the disclosure. In some examples, one or more of electronic devices 100a, 100b, 100c and/or 100d can calculate the likelihood of a user experiencing a health event as described herein (e.g., using the environmental and/or physiological data gathered by electronic devices 100a, 100b, 100c, and/or 100d). In some examples, electronic devices 100a, 100b, 100c, and/or 100d can generate an alert in response to calculating the likelihood of the user experiencing the health event exceeds a predetermined threshold.

FIG. 1A illustrates an example wearable device 100a (e.g., a watch or wristband). Wearable device 100a can include touch screen 102, crown 104, button 106, and strap 108. In some examples, the wearable device 100b can display a user interface including touch screen 102. In some examples, the wearable device 100b can receive user inputs (e.g., touch inputs) directed to touch screen 102 and perform operations in response to receiving the user inputs. Additionally or alternatively, in some examples, the wearable device 100a can receive user inputs using crown 104 and/or button 106 and can perform operations in response to receiving user inputs using crown 104 and/or button 106. In some examples, while a user uses wearable device 100a, the user attaches the wearable device 100a to their body (e.g., their wrist) using strap 108.

FIG. 1B illustrates an example earbud 100b. Earbud 100b can include tip 109, touch sensor 110, and microphone 111. In some examples, the user can insert tip 109 of earbud 100b into the car to use earbud 100b. For example, earbud 100b can be used with a second earbud (not shown), so that the user wears an earbud in each car during use. In some examples, earbud 100b can receive inputs using touch sensor 110 and can perform operations in response to receiving user inputs using the touch sensor 110. In some examples, earbud 100b can include a microphone 111. In some examples, earbud 100b can output audio, such as audio playing on another electronic device, such as wearable device 100a, headset 100c, smartphone 100d, or another electronic device.

FIG. 1C illustrates an example headset 100c. Headset 100c can include eyepiece 112, camera 113, strap 114, and crown 116. In some examples, eyepiece 112 can include a display generation component that the headset 110c can use to display a user interface. In some examples, headset 100c uses camera 113 to capture one or more still and/or video images of a physical environment of the headset 100c. For example, the headset 110c uses the display generation component to display extended reality (XR) and/or virtual reality (VR) content, including displaying portions of the physical environment captured with camera 113 and/or making portions of the physical environment visible through transparent portions of eyepiece 112. In some examples, while using the headset 100c, the user attaches the headset 100c to their head using strap 114. In some examples, the headset 100c receives inputs with crown 116 and, in response to receiving the inputs, performs one or more functions. Additionally or alternatively, in some examples, the headset 100c receives inputs using one or more cameras (e.g., camera 113), including detecting the user performing gesture(s) with their fingers, hands, and/or arms and/or detecting a location of the user's gaze.

FIG. 1D illustrates an example smartphone 100d. In some examples, smartphone 100d includes a touch screen 120, one or more cameras 118, and a button 122. In some examples, smartphone 100d uses touch screen 120 to display a user interface. In some examples, the smartphone 100d uses touch screen 120 to receive inputs and, in response to receiving inputs, performs operations. In some examples, additionally or alternatively, the smartphone 100d receives inputs using button 122 and, in response to receiving the inputs, performs operations. In some examples, the smartphone captures one or more (e.g., still or video) images using the one or more cameras 118.

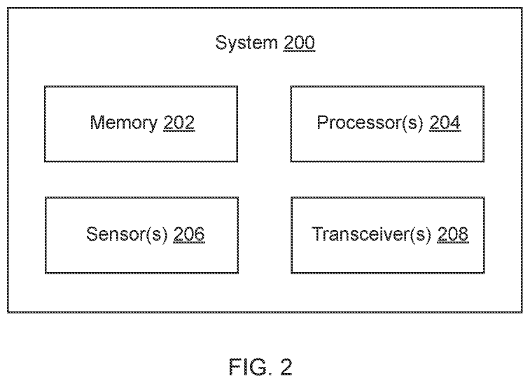

In some examples, wearable device 100a, earbud 100b, headset 100c, and/or smartphone 100d include one or more sensors additionally or alternatively to audio or optical sensors (e.g., microphone 111 and/or camera(s) 113 and 118), as described in more detail in the context of FIG. 2. Additionally or alternatively, in some examples, one or more of these example devices are in communication with one another and/or with one or more additional sensors. One or more of these devices can use the sensors to sense data about the physical environment of the device and/or physiological data of the user. In some examples, as described in more detail below, the raw sensor data and/or processed environmental data and/or physiological data can be obtained and processed by one or more of these devices (or another device), used to calculate the likelihood that the user of the device is experiencing or likely to experience a health event, such as a seizure. It should be understood that additional and/or alternative devices can be used similarly to electronic devices 100a, 100b, 100c, and/or 100d without departing form the scope of the disclosure, including but not limited to in-bed sleep tracking devices, personal computers, tablet computers, and/or media players.

FIG. 2 illustrates an example system 200 according to some examples of the disclosure. In some examples, system 200 corresponds to the basic architecture of one of the devices described above with reference to FIGS. 1A-1D. In some examples, system 200 corresponds to the basic architecture of more than one of the devices described above with reference to FIGS. 1A-1D. Additional or alternative components can be included in system 200 without departing from the scope of the disclosure.

In some examples, system 200 includes memory 202. Examples of memory 202 include random access memory (RAM), read-only memory (ROM), and/or flash memory, but other types of memory can be used without departing from the scope of the disclosure. In some examples, system 200 uses memory 202 to store instructions for completing the operation(s) and/or method(s) disclosed herein and/or to store data, such as user data and/or location-based data.

In some examples, system 200 includes processor(s) 204. Examples of processors include central processing units (CPUs), graphics processing units (GPUs), and/or neural processing units, but other types of processors can be used without departing from the scope of the disclosure. In some examples, processor(s) 204 include circuitry such as control unit(s), arithmetic logic unit(s), and/or register(s). In some examples, system 200 uses processor(s) 204 to perform operation(s), method(s), and/or calculation(s) disclosed herein.

In some examples, system 200 includes sensor(s) 206. Example sensors include location sensors (e.g., global positioning system (GPS)), optical sensors (e.g., light sensors, cameras), motion sensors (e.g., inertial measurement units (IMUs), electromyography (EMG) sensors, and/or cameras), eye tracking devices (e.g., electrooculography (EOG) sensors, EMGs, cameras), echocardiogram (ECG) sensors, photoplethysmography (PPG) sensors, pulse oximeter sensors, and/or audio sensor(s), but other types of sensors can be used without departing from the scope of the disclosure. In some examples, system 200 uses sensor(s) to sense data, such as environmental data and/or physiological data of a user of the system 200. As described herein, the system 200 uses the data to calculate a likelihood that the user of the system 200 is experiencing or is about to experience a health event, such as a seizure.

In some examples, system 200 includes transceiver(s) 208. Example transceivers include antennas that communicate using Wi-Fi, Bluetooth, and/or near-field communication (NFC), though other transceiver(s) and/or network types are possible without departing from the scope of the disclosure. In some examples, system 200 uses transceiver(s) 208 to communicate with other systems and/or electronic devices. For example, the system 200 uses the transceiver(s) 208 to obtain location-based data from a server and/or to send an alert to another system 200 when the system 200 calculates that the likelihood of the user of system 200 experiencing a health event is above a threshold likelihood.

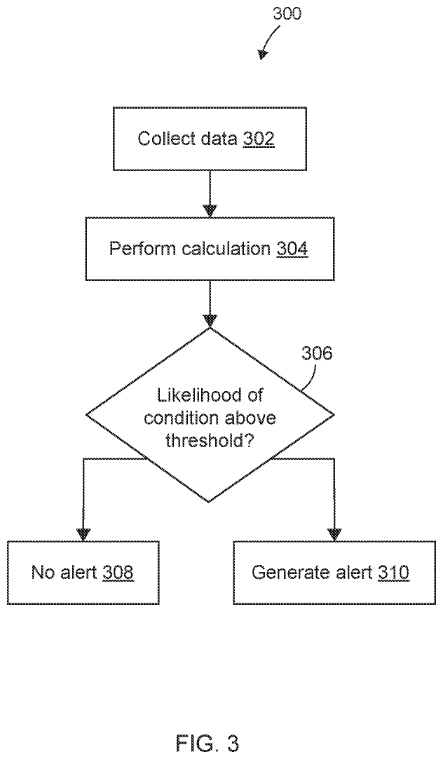

FIG. 3 illustrates an example method 300 of calculating the likelihood that a user is experiencing or is about to experience a health event according to some examples of the disclosure. In some examples, system 200, which can include one or more of the electronic devices 100a, 100b, 100c, and/or 100d (or other electronic devices) described above with reference to FIGS. 1A-1D, can perform method 300. For example, system 200 uses method 300 to calculate the likelihood that the user of the system 200 is having or is about to have a seizure. In some examples, determining the likelihood that the user is about to have a seizure includes determining a likelihood that the user will have a seizure within a threshold amount of time. In some examples, the threshold amount of time can vary depending on the stimulus detected, and can be in the range of milliseconds to minutes (e.g., 30, 60, 100, or 500 milliseconds; 1, 2, 3, 15, or 30 seconds; or 1, 2, or 5 minutes). Examples described with reference to FIG. 3 can apply to methods of detecting that the user is actively experiencing the health event, methods of predicting that the user is about to experience the health event, or both.

In some examples, method 300 includes collecting data (302). For example, system 200 uses sensor(s) 206 to collect data, including environmental data and/or physiological data of the user and/or data related to the user's daily routine, as described in more detail below. In some examples, environmental data includes data pertaining to the physical environment of system 200 as described herein. In some examples, physiological data of the user includes measurement of the user's body functions as described herein. In some examples, data related to the user's daily routine includes data sensed by one or more sensors and/or data reported by the user. In some examples, the data collected is customized to the user of system 200. For example, if the user has a diagnosis or history of experiencing adverse health events in response to particular triggers, data related to those triggers can be included in the calculation and/or weighted above other data. As another example, if the user has a diagnosis or history of demonstrating particular symptoms during or before experiencing adverse health events, data related to those symptoms can be included in the calculation and/or weighted above other data.

In some examples, the system 200 collects the data in response to the user activating a feature for calculating the likelihood of the health event. In some examples, the user is able to configure other aspects of this feature, including setting the threshold likeliness and opting in to various actions for the system 200 to take in response to detecting the likelihood of the user experiencing the health event exceeding the threshold, as described in more detail below.

In some examples, collecting the data includes adjusting the frequency of data sampling. In some examples, in response to receiving data that indicates an increased likelihood of the user experiencing the health event without exceeding the predefined likelihood threshold, the system 200 increases the sampling frequency. For example, in response to detecting environmental data that indicates that the environment of the system 200 could trigger the health event, the system 200 increases the sampling frequency of physiological data of the user. In some examples, while sensing data with the increased frequency, in response to calculating that the likelihood of the user experiencing the health event is not elevated (e.g., is below a threshold amount) and/or in response to determining the environment no longer includes the potential trigger, the system 200 reduces the sampling frequency. In some examples, the system 200 uses the increased sampling frequency for a predetermined period of time, such as around an hour from the first or last data indicating an increased likelihood of the user experiencing the health event.

Examples of environmental data include temperature data. In some examples, system 200 senses the temperature of the physical environment of the system 200 using a thermometer or other temperature sensors. In some situations, detecting environmental temperatures above a threshold temperature (e.g., 25, 28, or 30° C.) absolutely or for a threshold amount of time (e.g., 15, 30, or 45 minutes or 1, 2, 3, or 5 hours) can indicate increased likelihood that the user is about to experience a health event (e.g., seizure). As another example, some health events (e.g., seizure in reflex epilepsy) can be triggered by the user entering hot water (e.g., a bath, hot tub, or steam room). For example, the system 200 uses environmental temperature in a calculation of the likelihood that the user is experiencing or is about to experience a health event (e.g., seizure).

Examples of environmental data include light data. In some examples, system 200 uses light sensor(s) and/or camera(s) to sense the intensity, color, and/or pattern over time of light in the environment of the system 200. In some situations, light above a brightness threshold or light that changes intensities at a frequency over a threshold can trigger a seizure. For example, the system 200 uses environmental light data in the calculation of the likelihood that the user is experiencing or is about to experience a health event (e.g., seizure).

Examples of environmental data include sound data. In some examples, system 200 uses microphone(s) to sense the volume, pitch, timbre, and/or other characteristic(s) of sound in the environment of the system 200. In some situations, sound having volume above a predefined threshold (e.g., for an associated threshold time) and/or sound having a pitch in a predefined range can trigger adverse health events in sensitive individuals. For example, the system 200 uses environmental sound data in the calculation of the likelihood that the user is experiencing or is about to experience a health event (e.g., seizure).

In some examples, system 200 obtains additional environmental data from a server, such as environmental data collected by other electronic device(s) and/or other system(s). In some examples, the other electronic device(s) and/or other system(s) are associated with users other than the user of system 200. In some examples, users can opt in to share environmental data (e.g., temperature, light, and/or sound data) associated with respective locations that other electronic device(s) and/or system(s) can use to determine the likelihood that a user will experience a health event if the user goes to a location associated with the data.

In some examples, when the systems share environmental data with the system, the data is anonymized. For example, locations that frequently include environmental (e.g., light, sound, and/or temperature) conditions that may trigger a health event can be identified based on environmental data collected at those locations by electronic device(s) and/or system(s) that share collected data. Additionally or alternatively, in some examples, locations at which other users have frequently experienced health events triggered by the environment can be identified and anonymously shared by electronic device(s) and/or system(s) that share health event trigger data. In some examples, the server and/or system 200 constructs a “heat map” indicating the associations of various locations with increased likelihoods of triggering health events in sensitive individuals. In some examples, the server and/or system 200 customizes the “heat map” based on which data are relevant to the user depending on a diagnosis of the user and/or the user's history with experiencing adverse health events. For example, a “heat map” of lighting conditions is more relevant to a person with photosensitive epilepsy and/or a history of seizures triggered by lighting conditions than it is to a person without photosensitive epilepsy and no history of seizures triggered by lighting conditions.

In some examples, system 200 can use a location sensor to identify the current location of system 200. Optionally, the system 200 can transmit an indication of its current location to a server and receive, from the server, an indication of environmental data associated with the location collected by other device(s) and/or system(s). In some examples, the environmental data is anonymized. In some examples, the data is associated with a particular time of day. For example, lighting conditions may change throughout the day, and it is possible that a respective location will pose a higher risk of triggering a health event at some times of day than is the case at other times of day. For example, a wind turbine may cast shadows that create a flashing light effect at varying locations across different times of day, and may not create this effect at night. As another example, artificial lighting, such as marquees or other flashing lights, may stimulate a health event at night, but not during the day. In some examples, the system 200 can use location-based data to generate an alert when the likelihood of the user experiencing the health event exceeds the threshold. Additionally or alternatively, in some examples, the system 200 can use location-based data to adjust navigation directions to avoid directing the user through a physical area that could expose the user to health event triggers. In some examples, the system 200 can monitor environmental data, including location-based data, without monitoring physiological data of the user.

In some examples, the system 200 can sense and/or receive additional or alternative location-based data without departing from the scope of the disclosure. Moreover, in some examples, the system 200 can use this additional and/or alternative location-based data to calculate the likelihood of the user experiencing the health event as described herein.

Examples of physiological data include heart rate and/or heart rate variability. In some examples, system 200 senses heart rate and/or heart rate variability using ECG and/or PPG. For example, a wearable device, such as wearable device 100a includes a heart rate sensor on a surface of wearable device 100a in contact with the user's wrist while the user is using wearable device 100a. In some situations, detecting a heart rate elevated above a baseline, or manifestation of tachycardia for individuals who are non-tachycardic at baseline, can indicate an increased likelihood that the user is about to experience or is experiencing a health event (e.g., seizure). In some examples, the system 200 can also sense the duration of time during which the user's heart rate is elevated, and use the duration of time of elevated heart rate in the calculation of the likelihood that the user is experiencing or is about to experience the health event (e.g., seizure).

Examples of physiological data include body temperature of the user. In some examples, system 200 senses body temperature of the user with a thermometer or other temperature sensor. For example, wearable device 100a, earbud 100b, and/or headset 100c includes a thermometer positioned to sense the temperature of the body of the user while wearable device 100a, earbud 100b, and/or headset 100c is in use. As another example, system 200 is in communication with another temperature sensor in contact with the body of the user. In some situations, detecting an elevated body temperature (e.g., hyperthermia or fever) of the user can indicate an increased likelihood that the user is experiencing or is about to experience a health event (e.g., seizure). For example, fever can trigger a seizure and/or the user's body temperature may rise due to the seizure. In some examples, the system 200 senses metrics related to body temperature such as a binary determination of body temperature over a high threshold (e.g., 37, 37.5, 37.7, or 38° C.) or under a low threshold (e.g., 36° C.), absolute temperature, and/or duration of time for which the body temperature is over the high threshold or under the low threshold.

Examples of physiological data include body motion, including head, neck, and/or limb motion of the user of system 200. In some examples, system 200 senses body motion using one or more motion sensors (e.g., IMU and/or EMG) and/or one or more cameras. In some examples, the system 200 compares sensed body motion to predetermined motion patterns associated with the user experiencing the health event. For example, seizures can be characterized by jerking motion, so detecting motion data corresponding to jerking motion can indicate increased likelihood that the user is experiencing a seizure.

Examples of physiological data include oxygen saturation (SpO2). In some examples, the system 200 senses SpO2 of the user with a pulse oximeter sensor. For example, wearable device 100a includes a pulse oximeter sensor positioned to sense the wrist of the user while the user is using wearable device 100a. As another example, the system 200 is in communication with an additional or alternative pulse oximeter sensor (e.g., a finger worn pulse oximeter). In some examples, reduced SpO2 can indicate an increased likelihood that the user is experiencing a health event (e.g., seizure). In some examples, the system 200 can use detection of decreased SpO2 by a threshold amount (e.g., 15%, 20%, or 25%) and/or rectification of SpO2 after decreased SpO2 as an indication of increased likelihood the user is experiencing a health event.

Examples of physiological data include eye tracking data. In some examples, system 200 uses a camera or other eye tracking device to detect gaze motion, rate(s) of blinking, change(s) to the rate(s) of blinking, and/or pupil dilation among other possible characteristics. For example, headset 100c includes one or more eye tracking sensors positioned to capture eye tracking data while the user is using headset 100c. As another example, system 200 uses a different eye tracking device to collect eye tracking data (e.g., EOG, EMG, cameras etc.). In some examples, rapid blinking or staring (e.g., reduced rate of blinking) can indicate that a person is having a seizure. For example, if the system 200 detects absence of blinking for a threshold time (e.g., 2, 3, 5, 10, 15, or 30 seconds), rapid blinking above a threshold rate of blinking, and/or vertical and/or horizontal drift in eye position, the system 200 can calculate an increased likelihood that the user is experiencing a health event (e.g., seizure).

Examples of physiological data include electroencephalogram (EEG) data indicative of brain activity. In some examples, system 200 uses EEG sensors to detect brain activity of the user. In some examples, system 200 can process EEG data in the time-domain and/or frequency domain to determine whether EEG data indicates the user is likely experiencing or is about to experience a health event (e.g., seizure).

Examples of physiological data include vasodilation. For example, for some users, nitric oxide production can be elevated during seizures, which can contribute to vasodilation. Thus, for example, detecting vasodilation can indicate increased likelihood that the user is experiencing a seizure. In some examples, vasodilation can be more likely to occur in users that experience apnea, so the system 200 can adjust the weight of vasodilation in the calculation based on whether or not the user previously experienced apnea, which can contribute to hypoxia (which also agrees with decreased SpO2). In some examples, system 200 can measure vasodilation using ultrasonic imaging and/or gauge-strain plethysmography.

Examples of physiological data can include blood pressure. In some examples, seizures can lead to paroxysmal hypertension, a phenomenon where the blood pressure increases abruptly and significantly. For example, in response to detecting an increased blood pressure by a threshold amount (e.g., from 138/95 mmHg to 222/150 mmHg) in less than a threshold time (e.g., 10 seconds), the system 200 can determine that there is an increased likelihood that the user is experiencing a seizure. In some examples, an increase in blood pressure in this way accompanied by an increase in heart rate by a threshold amount (e.g., 20, 30 or 40 beats per minute (BPM)) or to a threshold level (e.g., 90, 100, 110, or 120 BPM) can indicate the increased likelihood that the user is experiencing a seizure, so the system 200 can evaluate these data together in performing the calculation. In some examples, the system 200 can use a blood pressure cuff or a cuffless blood pressure monitor to sense blood pressure.

Examples of physiological data include galvanic skin response. In some examples, electrodermal activity can increase during a seizure, so sensing increased electrodermal activity can indicate an increased likelihood that the user is experiencing a seizure. As another example, sensing galvanic skin response can indicate how much a user is sweating, and increased sweating can indicate increased likelihood the user is experiencing a seizure.

Examples of physiological data include respiration rate. For example, some patients experience apnea as a symptom of seizure. As another example, some patients experience hyperventilation as a symptom of seizure. Thus, in some examples, detecting either of these respiration rate patterns can indicate an increased likelihood that the user is experiencing a seizure. In some examples, the system 200 can use health information about the user, such as a diagnosis or self-reported information, such as previously-experienced symptoms during health events, to determine whether hyperventilation or apnea is a likely symptom of the health event. For example, for a user with a history of apnea as a symptom of seizure, detecting apnea could indicate an increased likelihood of the user experiencing the health event. As another example, for a user with a history of hyperventilation as a symptom of seizure, detecting hyperventilation could indicate an increased likelihood of the user experiencing the health event.

Additional and/or alternative examples of physiological data are possible without departing from the scope of the disclosure. The system 200 can use various sensors to measure these characteristics and can use data pertaining to these characteristics in part to calculate the likelihood of the user experiencing the health event.

In some examples, the system 200 can assess multiple characteristics together when calculating the likelihood that the user is experiencing or may be about to experience a health event. For example, co-occurrence of tachycardia and a decrease in SpO2 can more strongly indicate increased likelihood of the health event than occurrence of one of these symptoms without the other. As another example, brain activity that correlates to fluctuations in ambient light brightness can indicate the user is having or is about to have a health event.

In some examples, collecting physiological data includes collecting baseline measurements of one or more characteristics to be monitored for calculating the likelihood that the user is experiencing or is about to experience a health event. For example, the system 200 collects data for one or more of the physiological characteristics described above while the user is not experiencing the health event. In some examples, the system 200 collects baseline data multiple times (e.g., periodically, in response to a trigger, in response to a user request, etc.). As described in more detail below, the system 200 receives user feedback indicating whether the user has or has not experienced the health event. In some examples, in response to receiving feedback that the user has not experienced the health event for a period of time, the system 200 can classify physiological data collected during that period of time as baseline data. When monitoring the user's physiological characteristics to calculate the likelihood that the user will experience the health event, the system 200 can compare the collected data to the baseline data.

Additionally or alternatively, in some examples, the system 200 uses data related to the user's daily routine to calculate the likelihood that the user may experience a health event or is experiencing a health event. For example, menstruation, stress, alcohol consumption, sickness, and/or missed medication can increase the likelihood that a person experiences a health event (e.g., seizure). In some examples, system 200 can collect data related to these characteristics and determine when a user's regular routine is disrupted in a way that may increase the likelihood of the user experiencing a health event (e.g., seizure). For example, one or more electronic devices included in system 200 include applications for the user to log their health information, including data related to the characteristics listed previously. In some examples, the system 200 accesses this data and uses this data to calculate the likelihood of the user experiencing the health event.

Method 300 can include performing a calculation of the likelihood that the user of the system 200 is experiencing or is about to experience a health event (304). In some examples, the system 200 can use the data described above in calculating the likelihood that the user is experiencing a health event or will experience the health event. As described above, in some examples, the system 200 customizes the particular data used, the relative weights of different types of data, and the calculations performed based on a diagnosis or history of health events of the user.

In some examples, the calculation can include applying a variety of weights to different data. The weights can be based on health information about the user, including diagnoses of the user included in medical chart data and/or data related to previous health incidents provided to the system 200, as described in more detail below. For example, the system 200 will assign more weight to light data if the user has a diagnosis of photosensitive epilepsy than would be the case if the user did not have a diagnosis of photosensitive epilepsy. As another example, the system 200 will assign more weight to ECG data if the user previously had a health incident correlated with irregular ECG data than would be the case if the user did not previously have a health incident correlated with irregular ECG data. In some examples, the calculation can include a classifier algorithm, a neural network, and/or other machine learning techniques. In some examples, machine learning techniques can be implemented in hardware (e.g., using one or more processors configured for machine learning) and/or software (e.g., one or more programs including instructions for executing the machine learning algorithm). For example, the system 200 uses binary regression for probabilistic classification of the likelihood that the user of the system 200 is experiencing or is about to experience a health event.

Method 300 can include determining whether the calculated likelihood that the user is experiencing the health event or will experience the health event exceeds a predetermined threshold (306). In some examples, the threshold is a unitless number resulting from the calculation based on the characteristics described above. The threshold can be an absolute likelihood or a threshold increase of a baseline likelihood, for example. In some examples, the threshold is an absolute likelihood or an increased likelihood of 20%, 30%, 50%, or 75%. If the likelihood exceeds the threshold, the system 200 can generate an alert (310). In some examples, the user can customize the threshold likelihood at which the system 200 will generate the alert. In some examples, generating the alert can include performing one or more actions. In some examples, the system 200 can set different likelihood thresholds associated with different actions as described in more detail below. In some examples, the threshold is based on balancing the likelihoods of false positives (predicting a health event when one does not occur) versus false negatives (failing to predict a health event). In some situations, users may prefer to reduce the likelihood of false negatives at the expense of the calculation resulting in an increased number of false positives. For example, the system 200 may use a threshold that generates no missed health events and around one false positive per week or less. In some examples, the user is able to input a number of false positives they would find acceptable and a number of false negatives they would find acceptable, and the system 200 can design a threshold based on the user's preferences.

For example, generating the alert can include notifying the user of the system 200 that the likelihood of the user experiencing the health event exceeds the threshold. In some examples, the system 200 notifies the user by outputting a visual, audio, and/or tactile indication with one or more output devices. In some examples, outputting the indication includes displaying a user interface element for providing feedback to the system 200, such as feedback that the user is not experiencing the health event or feedback that the user is experiencing the health event and is safe. In some examples, the indication includes a selectable option that, when selected, causes the system 200 to contact an emergency contact of the system 200, emergency services (e.g., police and/or an ambulance), and/or good Samaritans nearby with an indication that the user is experiencing the health event. In some examples, good Samaritans include users of other electronic device(s) and/or systems that have training to assist when a person is experiencing a health incident, such as first aid certification and/or training as a healthcare professional (e.g., medical technicians, nurses, and/or doctors) and have opted in to being alerted if another person is experiencing or likely to be experiencing an adverse health event. In some examples, the system 200 notifies good Samaritans within a threshold distance (e.g., 50, 100, 200, 300, 500, or 750 meters or 1, 2, 3, or 5 kilometers) of the system 200 when the indication is generated. In some examples, a system 200 in use by a child can contact a parent or guardian of the child in response to the calculation exceeding the threshold. For example, the parent or guardian can select the threshold likelihood that the child is experiencing a health event at which they would like to be notified. In some examples, the system 200 can notify the parent or guardian of the child and/or the user of system 200 when exposure to a stimulus has occurred, even if the health event does not occur. In some examples, the system 200 outputs escalating indications to the user of system 200 if a response to an indication is not received in a threshold amount of time (e.g., 5, 10, 15, 30, or 45 seconds or 1, 2, 3, 5, 10, 15, or 20 minutes). For example, the system outputs a tactile (e.g., haptic) indication with increasing intensities if user input is not received. As another example, if the system 200 does not receive a response after outputting one or more tactile indications, the system 200 outputs an audio indication additionally or alternatively to the tactile indication.

In some examples, if the system 200 does not receive a user input responding to the indication that the likelihood of the health event occurring exceeds the threshold within a predetermined amount of time (e.g., 5, 10, or 30 minutes), the system 200 notifies another electronic device and/or system 200, such as an electronic device and/or system used by the emergency contact of the user, emergency services, and/or good Samaritans nearby. In some embodiments, the system 200 transmits the indication to the other system without receiving an input from the user. In some examples, the system 200 includes a settings user interface with which the user is able to interact to change a setting for which systems should be contacted if the likelihood of the health event exceeds the threshold amount. In some embodiments, the user is able to disable the system 200 from notifying other systems and/or electronic devices that the likelihood of the health event exceeds the threshold. In some embodiments, the system 200 transmits medical information about the user, previously authorized by the user (e.g., in the settings user interface) before the system calculated that the health event was likely, to the other system and/or electronic devices, such as the user's name, allergies, diagnoses related to the health event, medical insurance information, and the like. In some examples, the system 200 displays this health information when the indication is generated. In some examples, the system 200 displays and/or transmits to the emergency contact, emergency services, and/or good Samaritans information about the generated indication, such as the symptoms and/or triggers detected by the system 200 and/or relevant diagnoses and/or previous incidents of the user. In some examples, the system 200 transmits an indication of the current location of the system 200 to the emergency contact, emergency services, and/or good Samaritans when transmitting the indication.

In some examples, the user can customize the threshold likelihood needed to trigger respective actions associated with the alert. For example, the system 200 may be configured to notify the user, but not notify other systems, if the likelihood of the health event is above a low threshold but not above a high threshold. As another example, the system 200 may be configured to notify other systems if the likelihood of the health event is above the high threshold and, optionally, the user does not respond to an indication requesting user feedback.

Additionally or alternatively, in some examples, the system 200 can generate an alert in response to detecting a stimulus of the health event even if the threshold likelihood of the user experiencing the health event is below the threshold likelihood. For example, the system 200 can present an audio, visual, and/or tactile indication that includes an indication of what the stimulus was and/or a degree to which the user was exposed (e.g., intensity and/or duration of the stimulus).

Additionally or alternatively, in some examples, the system 200 can modify operation of one or more devices that impact the user's environment in response to calculating that the likelihood of the user experiencing the health event exceeds the threshold and/or in response to other factors that could trigger the user into experiencing the health event. For example, if the user is sensitive to flashing lights, the system 200 can take steps to limit the user's exposure to flashing lights when watching video content that may trigger a health event in the user. In some examples, while an electronic device in communication with the system 200 or included in the system is playing a content item, and the electronic device and/or system 200 determines that an upcoming portion of the content item includes flashing lights, the system 200 can take actions to decrease the triggering aspect of the content item. For example, the system 200 can adjust other lights in the environment of the user (e.g., smart lamps or other smart lighting) to increase the ambient light levels, thereby decreasing the amount of contrast in ambient light levels caused by the content item. As another example, the system 200 can reduce the image size of the content item during the portion that includes flashing lights. In some examples, if the user can be triggered by sounds with a particular characteristic (e.g., loud, sudden, and/or high-pitched sounds), the system 200 can reduce the playback volume of the content item during the portion including such sounds. Additionally or alternatively, in some examples, the system 200 can skip portions of the content item including images and/or sounds that may trigger the user's health condition.

In some examples, the method includes, if the likelihood of the health event is below the threshold forgoing generating the alert (308). As described above, in some examples, the system 200 collects and/or stores data not associated with the user experiencing health events as baseline data that can be used in the future to calculate the likelihood of the health event. As described below, the system 200 can also update the algorithm used in subsequent calculations in accordance with receiving user feedback related to the health event.

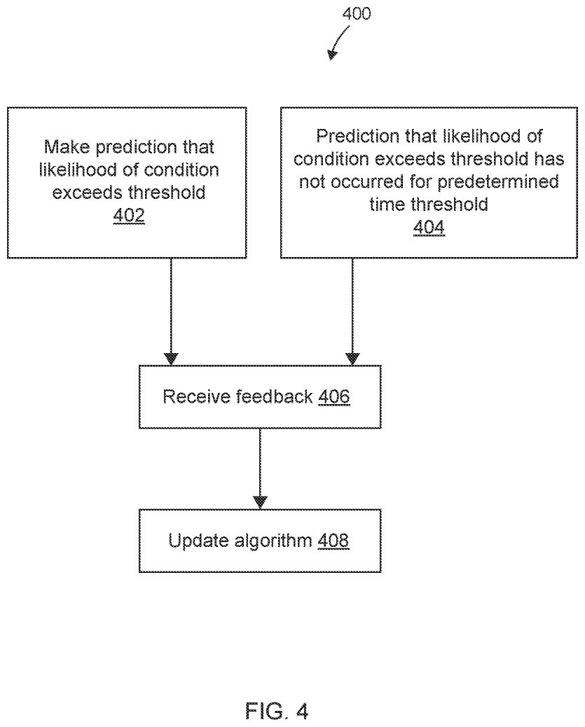

FIG. 4 illustrates an example method 400 of updating an algorithm used to calculate the likelihood that a user of a system 200 is experiencing a health event or is about to experience a health event according to some examples of the disclosure. In some examples, method 400 is performed by system 200 to improve the algorithm used in method 300 to calculate the likelihood of the user experiencing the health event. Examples described with reference to FIG. 4 can apply to methods of detecting that the user is actively experiencing the health event, methods of predicting that the user is about to experience the health event, or both.

In some examples, method 400 includes making a prediction that the likelihood of the user experiencing the health event exceeds a predetermined threshold (402). In some examples, the system 200 makes this prediction using method 300 described above. In some examples, method 400 includes receiving user feedback (406). For example, as described above at least with reference to FIG. 3, in response to determining that the likelihood of the user experiencing the health event exceeds the threshold, the system 200 generates an indication that can include one or more selectable options for the user to provide feedback to the system. For example, the user can confirm they are experiencing the health event or indicate that they are not experiencing the health event. In some examples, the user can provide feedback about a received alert after receiving the alert, such as hours or days later. In some examples, the system 200 keeps a record of generated alerts and/or confirmed health events using a health record application. In some examples, in response to receiving an indication that the user did not experience the health event, the system 200 can label the data as not corresponding the health event. In some examples, in response to receiving an indication that the user experienced the health event, the system 200 can label the data as corresponding the health event.

In some examples, method 400 includes updating the algorithm (408). For example, the system 200 updates the algorithm in accordance with the user feedback. In some situations, the system 200 may calculate that the likelihood of the user experiencing the health event exceeds the threshold but receive user feedback that the user did not experience the health event. In some examples, in response to these false positive predictions, the system 200 can update the algorithm to avoid generating a false positive in response to similar data in the future. For example, the system 200 may adjust the relative weights of various types of data, add and/or remove data from consideration, and/or adjust the operations performed on the data to avoid a false positive result in response to similar data in the future. For example, the system 200 can decrease the weight assigned to a category of data that indicated an increased likelihood of the user experiencing a health event or remove the category of data from future consideration. As another example, the system 200 can increase the weight assigned to a category of data that indicated a decreased likelihood of the user experiencing the health event and/or add the category of data to consideration if it was not already being considered.

In some situations, the system 200 may calculate that the likelihood of the user experiencing the health event exceeds the threshold and receive user feedback that the user was indeed experiencing the health event. In some examples, in response to these true positive predictions, the system 200 can update the algorithm to improve performance in the future, such as to be able to make the prediction more quickly in response to similar data patterns. For example, the system 200 may adjust the relative weights of various types of data, add and/or remove data from consideration, and/or adjust the operations performed on the data to avoid a false positive result in response to similar data in the future. For example, the system 200 can decrease the weight assigned to a category of data that indicated a decreased likelihood of the user experiencing a health event or remove the category of data from future consideration. As another example, the system 200 can increase the weight assigned to a category of data that indicated an increased likelihood of the user experiencing the health event and/or add the category of data to consideration if it was not already being considered.

In some examples, method 400 includes making a prediction that the likelihood of the user experiencing the health event is less than a predetermined threshold for a predetermined time threshold (404). For example, the predetermined time threshold can be 1, 3, or 5 days, 1 or 2 weeks, or a month. In some examples, the system 200 makes this prediction using method 300 described above. In some examples, method 400 includes receiving user feedback (406). For example, in response to making the prediction that the likelihood of the user experiencing the health event was less than the likelihood threshold for at least the threshold amount of time, the system 200 generates an indication that can include one or more selectable options for the user to provide feedback to the system. For example, the user can confirm they did not experience the health event during the predetermined period of time. In some examples, in response to receiving an indication that the user did not experience the health event for the period of time, the system 200 can label the data collected during the period of time as not corresponding the health event (e.g., baseline data). In some examples, the system 200 includes an application into which the user can enter inputs indicating that they are experiencing or have recently experienced the health event without receiving the indication that the likelihood of the user experiencing the health event exceeds the threshold. For example, if the user experiences the health event without the system 200 predicting the health event based on the data, the user can provide feedback to the system indicating when they experienced the health event. In this example, in response, the system 200 can label the data from the time during which the user was experiencing the health event as corresponding to the health event.

In some examples, method 400 includes updating the algorithm (408). For example, the system 200 updates the algorithm in accordance with the user feedback. In some situations, the system 200 may calculate that the likelihood of the user experiencing the health event was less than the threshold but receive user feedback that the user experienced the health event. In some examples, in response to these false negative predictions, the system 200 can update the algorithm to avoid generating a false negative response to similar data in the future. For example, the system 200 may adjust the relative weights of various types of data, add and/or remove data from consideration, and/or adjust the operations performed on the data to avoid a false negative result in response to similar data in the future. For example, the system 200 can decrease the weight assigned to a category of data that indicated a decreased likelihood of the user experiencing a health event or remove the category of data from future consideration. As another example, the system 200 can increase the weight assigned to a category of data that indicated an increased likelihood of the user experiencing the health event and/or add the category of data to consideration if it was not already being considered.

In some situations, the system 200 may calculate that the likelihood of the user experiencing the health event was less than the threshold for the predetermined time period and receive user feedback that the user did not experience the health event for the predetermined time period. In some examples, in response to these true negative predictions, the system 200 can update the algorithm to improve performance in the future, such as to be able to make the prediction more quickly in response to similar data patterns. For example, the system 200 may adjust the relative weights of various types of data, add and/or remove data from consideration, and/or adjust the operations performed on the data to avoid a false positive result in response to similar data in the future. For example, the system 200 can decrease the weight assigned to a category of data that indicated an increased likelihood of the user experiencing a health event or remove the category of data from future consideration. As another example, the system 200 can increase the weight assigned to a category of data that indicated a decreased likelihood of the user experiencing the health event and/or add the category of data to consideration if it was not already being considered.

In some examples, other techniques of updating the algorithm are possible. For example, the system 200 can use machine learning to make predictions and/or to update the algorithm. As another example, in response to receiving additional health data about the user, such as clinical test results and/or diagnoses, the system 200 can update the algorithm based on characteristics associated with the test results and/or diagnoses. In some examples, the system 200 can use the collected data together with user feedback labeling the data as correlated to the health event or not correlated to the health event to train a machine learning algorithm or to refine the machine learning algorithm.

Aspects of the disclosure relate to collection and, in some circumstances, sharing of sensitive information, such as health information and/or location information as disclosed herein. This information is to be handled in a manner that preserves and respects privacy of the user, meeting or exceeding privacy standards established by industry and/or government regulation. Techniques for protecting privacy include, but are not limited to, encrypting the information, anonymizing information before sharing, and/or allowing the user to opt-in/opt-out of the collection and sharing of information.

Some examples are directed to system comprising one or more sensors; one or more input devices; one or more output devices; and one or more processors configured to perform a method comprising: sensing, using the one or more sensors, physiological data of a user of the system and a current location of the system; in accordance with a calculation based on the physiological data and the current location of the system indicating that a likelihood of the user of the system experiencing a health event is above a threshold value: outputting, using the one or more output devices, a request for user feedback indicating whether or not the health event occurred; receiving, via the one or more input devices, the user feedback indicating whether or not the health event occurred; and updating the calculation for future use based on the user feedback. In some examples, the method further comprises: sensing, using one or more second sensors, environmental data of a physical environment of the system, wherein the calculation is further based on the environmental data. In some examples, the calculation is based on an association of the current location of the system with the health event. In some examples, the calculation is based on an association of the current location of the system and a current time with the health event. In some examples, the calculation is based on health data associated with the user of the system. In some examples, the method further comprises: performing the calculation with the system without performing the calculation with another system. In some examples, the method further comprises: in accordance with the calculation based on the physiological data and the current location of the system indicating that the likelihood of the user of the system experiencing the health event is above the threshold value, transmitting a notification to a second system associated with a second user different from the user of the system. In some examples, the method further comprises: in accordance with a determination that the likelihood of the user of the system experiencing the health event has been above the threshold value for at least a predetermined amount of time: outputting, using the one or more output devices, a request for second user feedback indicating whether or not the health event occurred; receiving, via the one or more input devices, the second user feedback indicating whether or not the health event occurred; and updating the calculation for future use based on the second user feedback. In some examples, the method further comprises: receiving, via the one or more input devices, second user feedback indicating that the health event occurred; and in response to receiving the second user feedback that the health event occurred, in accordance with a second calculation based on the physiological data and the current location of the system indicating that the likelihood of the user of the system experiencing the health event is below the threshold value, updating the calculation for future use. In some examples, the method further comprises: playing a content item using the one or more output devices; in accordance with a determination that an upcoming portion of the content item has a characteristic associated with an increased likelihood of the user of the system experiencing the health event: modifying a characteristic of an environment of the user using the one or more output devices in accordance with the characteristic associated with the increased likelihood of the user of the system experiencing the health event.

Some examples are directed to a non-transitory computer readable storage medium storing instructions that, when executed by a system including one or more sensors, one or more input devices, one or more output devices, and one or more processors, causes the system to perform a method comprising: sensing, using the one or more sensors, physiological data of a user of the system and a current location of the system; in accordance with a calculation based on the physiological data and the current location of the system indicating that a likelihood of the user of the system experiencing a health event is above a threshold value: outputting, using the one or more output devices, a request for user feedback indicating whether or not the health event occurred; receiving, via the one or more input devices, the user feedback indicating whether or not the health event occurred; and updating the calculation for future use based on the user feedback. In some examples, the method further comprises: sensing, using one or more second sensors, environmental data of a physical environment of the system, wherein the calculation is further based on the environmental data. In some examples, the calculation is based on an association of the current location of the system with the health event. In some examples, the calculation is based on an association of the current location of the system and a current time with the health event. In some examples, the calculation is based on health data associated with the user of the system. In some examples, the method further comprises: performing the calculation with the system without performing the calculation with another system. In some examples, the method further comprises: in accordance with the calculation based on the physiological data and the current location of the system indicating that the likelihood of the user of the system experiencing the health event is above the threshold value, transmitting a notification to a second system associated with a second user different from the user of the system. In some examples, the method further comprises, in accordance with a determination that the likelihood of the user of the system experiencing the health event has been above the threshold value for at least a predetermined amount of time outputting, using the one or more output devices, a request for second user feedback indicating whether or not the health event occurred; receiving, via the one or more input devices, the second user feedback indicating whether or not the health event occurred; and updating the calculation for future use based on the second user feedback. In some examples, the method further comprises: receiving, via the one or more input devices, second user feedback indicating that the health event occurred; and in response to receiving the second user feedback that the health event occurred, in accordance with a second calculation based on the physiological data and the current location of the system indicating that the likelihood of the user of the system experiencing the health event is below the threshold value, updating the calculation for future use. In some examples, the method further comprises playing a content item using the one or more output devices; in accordance with a determination that an upcoming portion of the content item has a characteristic associated with an increased likelihood of the user of the system experiencing the health event: modifying a characteristic of an environment of the user using the one or more output devices in accordance with the characteristic associated with the increased likelihood of the user of the system experiencing the health event.

Some examples are directed to a method comprising: at a system in communication with one or more sensors, one or more input devices, one or more output devices, and one or more processors: sensing, using the one or more sensors, physiological data of a user of the system and a current location of the system; in accordance with a calculation based on the physiological data and the current location of the system indicating that a likelihood of the user of the system experiencing a health event is above a threshold value: outputting, using the one or more output devices, a request for user feedback indicating whether or not the health event occurred; receiving, via the one or more input devices, the user feedback indicating whether or not the health event occurred; and updating the calculation for future use based on the user feedback.

Although the disclosed examples have been fully described with reference to the accompanying drawings, it is to be noted that various changes and modifications will become apparent to those skilled in the art. Such changes and modifications are to be understood as being included within the scope of the disclosed examples as defined by the appended claims.

1. A system comprising:
one or more sensors;
one or more input devices;
one or more output devices;
memory; and
one or more processors configured to perform a method comprising:
sensing, using the one or more sensors, physiological data of a user of the system and a current location of the system;
in accordance with a calculation based on the physiological data and the current location of the system indicating that a likelihood of the user of the system experiencing a health event is above a threshold value:
outputting, using the one or more output devices, a request for user feedback indicating whether or not the health event occurred;
receiving, via the one or more input devices, the user feedback indicating whether or not the health event occurred; and
updating the calculation for future use based on the user feedback.

2. The system of claim 1, wherein the method further comprises:
sensing, using one or more second sensors, environmental data of a physical environment of the system, wherein the calculation is further based on the environmental data.

3. The system of claim 1, wherein the calculation is based on an association of the current location of the system with the health event.

4. The system of claim 1, wherein the calculation is based on an association of the current location of the system and a current time with the health event.

5. The system of claim 1, wherein the calculation is based on health data associated with the user of the system.

6. The system of claim 1, wherein the method further comprises:
performing the calculation with the system without performing the calculation with another system.

7. The system of claim 1, wherein the method further comprises:
in accordance with the calculation based on the physiological data and the current location of the system indicating that the likelihood of the user of the system experiencing the health event is above the threshold value, transmitting a notification to a second system associated with a second user different from the user of the system.

8. The system of claim 1, wherein the method further comprises:
in accordance with a determination that the likelihood of the user of the system experiencing the health event has been above the threshold value for at least a predetermined amount of time:
outputting, using the one or more output devices, a request for second user feedback indicating whether or not the health event occurred;
receiving, via the one or more input devices, the second user feedback indicating whether or not the health event occurred; and
updating the calculation for future use based on the second user feedback.

9. The system of claim 1, wherein the method further comprises:
receiving, via the one or more input devices, second user feedback indicating that the health event occurred; and
in response to receiving the second user feedback that the health event occurred, in accordance with a second calculation based on the physiological data and the current location of the system indicating that the likelihood of the user of the system experiencing the health event is below the threshold value, updating the calculation for future use.

10. The system of claim 1, wherein the method further comprises:
playing a content item using the one or more output devices;
in accordance with a determination that an upcoming portion of the content item has a characteristic associated with an increased likelihood of the user of the system experiencing the health event:
modifying a characteristic of an environment of the user using the one or more output devices in accordance with the characteristic associated with the increased likelihood of the user of the system experiencing the health event.

11. A non-transitory computer readable storage medium storing instructions that, when executed by a system including one or more sensors, one or more input devices, one or more output devices, memory, and one or more processors, causes the system to perform a method comprising:
sensing, using the one or more sensors, physiological data of a user of the system and a current location of the system;
in accordance with a calculation based on the physiological data and the current location of the system indicating that a likelihood of the user of the system experiencing a health event is above a threshold value:
outputting, using the one or more output devices, a request for user feedback indicating whether or not the health event occurred;
receiving, via the one or more input devices, the user feedback indicating whether or not the health event occurred; and
updating the calculation for future use based on the user feedback.

12. The non-transitory computer readable storage medium of claim 11, wherein the method further comprises:
sensing, using one or more second sensors, environmental data of a physical environment of the system, wherein the calculation is further based on the environmental data.

13. The non-transitory computer readable storage medium of claim 11, wherein the calculation is based on an association of the current location of the system with the health event.

14. The non-transitory computer readable storage medium of claim 11, wherein the calculation is based on an association of the current location of the system and a current time with the health event.

15. The non-transitory computer readable storage medium of claim 11, wherein the calculation is based on health data associated with the user of the system.

16. The non-transitory computer readable storage medium of claim 11, wherein the method further comprises:
performing the calculation with the system without performing the calculation with another system.

17. The non-transitory computer readable storage medium of claim 11, wherein the method further comprises:
in accordance with the calculation based on the physiological data and the current location of the system indicating that the likelihood of the user of the system experiencing the health event is above the threshold value, transmitting a notification to a second system associated with a second user different from the user of the system.

18. The non-transitory computer readable storage medium of claim 11, wherein the method further comprises:
in accordance with a determination that the likelihood of the user of the system experiencing the health event has been above the threshold value for at least a predetermined amount of time:
outputting, using the one or more output devices, a request for second user feedback indicating whether or not the health event occurred;
receiving, via the one or more input devices, the second user feedback indicating whether or not the health event occurred; and
updating the calculation for future use based on the second user feedback.

19. The non-transitory computer readable storage medium of claim 11, wherein the method further comprises:
receiving, via the one or more input devices, second user feedback indicating that the health event occurred; and
in response to receiving the second user feedback that the health event occurred, in accordance with a second calculation based on the physiological data and the current location of the system indicating that the likelihood of the user of the system experiencing the health event is below the threshold value, updating the calculation for future use.

20. The non-transitory computer readable storage medium of claim 11, wherein the method further comprises:
playing a content item using the one or more output devices;
in accordance with a determination that an upcoming portion of the content item has a characteristic associated with an increased likelihood of the user of the system experiencing the health event:
modifying a characteristic of an environment of the user using the one or more output devices in accordance with the characteristic associated with the increased likelihood of the user of the system experiencing the health event.
